(19) United States
(12) Patent Application Publication
(10) Pub. No.: US 2011/0091581 A1
(43) Pub. Date: Apr. 21, 2011

(54) New pharmaceutical function of dichroa febrifuga alkone derivative (DFAD)

(76) Inventor: Xiao Qi Wang, Ningbo (CN)

(21) Appl. No.: 12/587,880

(22) Filed: Oct. 15, 2009

(51) Int. Cl. A61K 36/185; A61P 33/06

(52) U.S. Cl. 424/725

(57) ABSTRACT

A safe pharmaceutical composition for antioxidation, controlling oncoges, inhibiting tyrodine kinase activity and increasing immune function contains dichroa febrifuga alkone derivative (DFAD).

### DESCRIPTION OF PRIOR ART

The natural source of dichroa febrifuga alkone derivative (DFAD) is abound. DFAD is extracted from plant named dichroa febrifuga (Blue Evergreen Hydrangea). The plant has 4 to 8 inch long dark green resemble the foliage of Hydrangea with prominent veins and small serrations. The terminal end of the branches hold clusters of Hydrangea-like flowers with white buds opening to bright blue flowers in spring and summer that are followed by metallic blue berries. As with the blue forms of Hydrangea the shade of blue of the flower is determined by soil pH and more acid soils produce bluer flowers. Plant in part sun to light shade with moderately moist soil. It is hardy and evergreen to 20-25 degrees F. but defoliates such below these temperatures but plants knocked back by cold resprout from hard wood. Dichroa febrifuga is native to Nepal eastwards to southern China and into south-east Asia, where it grows at the forest edge. The specific epithet febrifuga is in reference to the use of the plant as a febrifuge, acting to reduce fever. Its use as such is reported in China.

Febrifugine was isolated from plant and synthesis. For example, febrifugines is isolated cis-febrifugine and trans-febrifugine from Hydrangea macrophylla (Saxifragaceae); these compounds had already been isolated from Dichroa febrifuga (antimalarial natural drug) by Koepfli et al. in 1947 and from Hydrangea umbellate by Ablondi et al. in 1952. Synthesis of febrifugins was first achieved in 1952 by Baker et al., who later reported that febrifugine obtained from D. febrifuga corresponds to the synthetic cis-febrifugine, whereas isofebrifugine corresponds to the synthetic trans-febrifugine. Afterwards, Barringer et al. established through detailed H-NMR analysis that the assignments by Baker et al. should be reversed, i.e., febrifugine has a trans-orientation and isofebrifugine has a cis-configuration. Thus, the absolute configurations of febrifugines were established.

Febrifugine and isofebrifugine derived from Chinese hydrangea are known to have strong activities against tropical malarial protozoan. The chemical structures of febrifugine and isofebrifugine are known to show such strong activities against malarial. The activity of these febrifugine compounds have been known from old times as active ingredients of Chinese medicines such as “JOSAN”.

### DETAILED DESCRIPTION

Malaria is one which of most serious infectious diseases. In the known infectious disease, it is only inferior to human health's harm to pulmonary tuberculosis. According to the World Health Organization reported that world has every 3 to 600,000,000 people infects malaria, dies the approximately 3,000,000 people. In 2005 the 58^{th} World Health Assembly pointed out: every year malaria continues the death which creates more than 100 ten thousand may prevent, particularly in Africa's babies and other frail crowds, and this disease continues to threaten the Americans, Asians and the Pacific section several million person of lives. Although the existing medicine (for example quinoline chloroquinoline and so on) has certain curative effect to malaria, but the human body will have the drug resistance rapidly in the course of treatment. As early as in ancient China, used the saxifragaceae Chinese herb dichroa febrifuga to use in the malaria shot.

The present invention disclosed new pharmaceutical function of DFAD.

The following specific examples will provide detailed illustrations of methods of producing relative drugs, according to the present invention and pharmaceutical dosages units containing relative drugs. Moreover, examples described pharmaceutical characters of drugs, which demonstrated its effectiveness in control of cancer cells. These examples are not intended, however, to limit or restrict the scope of the invention in any way, and should not be construed as providing conditions, parameters, reagents, or starting materials which must be utilized exclusively in order to practice the present invention.

### Example 1

### Effects of DFAD on Super-Oxidation During Reperfusion of Ischemia Heart

A number of evidences indicated that superoxide anion, hydrogen superoxide, and hydroxyl radical were directive causes for ischemia or reperfusion damage. H₂O₂ in the body is cleared by catalase and glutathione peroxidase (GSH-Px) catalysis. In myocardium, catalase activity of GSH-Px was lower.

In the present study we examined the effect of DFAD on super-oxidation during reperfusion of ischemia heart.

Male rats (280 to 320 g body weight) of Spague-Dawley strain maintained on a standard diet were used in these experiments. The rats were lightly anesthetized with diethyl ether, the left femoral vein was exposed and heparin (200 IU) was administered intravenously. 1 minute after administration of heparin the heart was excised and placed in ice cool perfusion medium until contraction had ceased. The heart was then mounted on the perfusion apparatus.

Langendorff perfusion for an initial 10 min. period, the perfusion was continued as a working preparation for an additional 30 min. The hearts were all electrically paced at between 265 and 275 beats/min during working perfusion. This working load resulted in 60% to 75% of maximal, maintainable peak systolic pressure.

The perfusate was Krebs-Henseleit bicarbonate buffer gassed with 95% oxygen and 5% carbon dioxide. This perfusate contained 11 mM D-glucose during the Langendorff perfusion.

Aortic pressure, heart rates, coronary flows and aortic outputs were monitored during perfusion. The perfusate of treatment group contained 0.005 mg of DFAD/ml. The perfusate of control group contained buffer only. At the end the frozen hearts were store in liquid nitrogen until assayed for metabolic intermediates.

The tissue sample was extracted in ice-cold 10% trichloroacetic acid and centrifuged at 10,000×g for 15 min. at 4° C. The precipitates of trichloroacetic acid were washes with 1% trichloroacetic acid and subsequently used for determining levels biochemical index of lactate. The data were expressed per gram of non-collagen protein (NCP).

SOD and GSH-Px were assayed.

The experimental data are listed in Table 1.

TABLE 1
Effects of DFAD on super-oxidation (1)

| Group | SOD activity (μg/protein) |
|---|---|
| Normal (N) | 11.0 ± 1.5 |
| Ischemia (C) | 7.0 ± 0.8 |
| Ischemia + DFAD (T) | 8.5 ± 1.2* |

*P < 0.05 as compared with C group.

TABLE 2
Effects of DFAD on super-oxidation (2)

| Group | GSH-Px (μ/g.w) |
|---|---|
| Normal (N) | 20.0 ± 2.5 |
| Ischemia (C) | 15.4 ± 1.5 |
| Ischemia + DFAD (T) | 17.5 ± 2.0* |

*P < 0.05 as compared with C group.

This study indicated when myocardium ischemia, SOD activity was low. When ischemia 60 min, a large number of free radicals produced in myocardium. It is also known that product of free radicals was more obvious in reperfusion time.

With ischemia time extension, GSH-Px activity started to lower. When ischemia 60 min, it was more lower. Pre-perfusion with 4×10⁻⁶ g of DFAD/ml, SOD and GSH-Px activities were significantly higher than those in control group. Data of Table 1 and 2 showed that DFAD could significantly reduce oxygen radical level of reperfusion ischemia heart.

### Example 2

### Effects of DFAD on Peroxidation

It is known that DFAD can inhibit lipid peroxidation.

The experiments were performed as previously described. Hepatic lipoperoxide content was determined as described by Uchiyama.

In the present study, the effect of DFAD in lipoperoxides was examined.

TABLE 3
Effects of DFAD on peroxidation

| | Lipoperoxides | | | | |
|---|---|---|---|---|---|
| | (mmol MDA/g liver protein) | | | | |
| Group | 1 h | 3 h | 6 h | 12 h | 24 h |
| Control | 0.80 ± 0.09 | 0.90 ± 0.09 | 1.1 ± 1.0 | 1.3 ± 1.2 | 2.2 ± 2.0 |
| Treatment | 0.72 ± 0.08* | 0.78 ± 0.09* | 0.9 ± 0.08* | 1.1 ± 0.4** | 1.7 ± 0.85* |

*P < 0.05 as compared with control group.

It is known that the pathogenesis of CCl₄—induced hepatic damage involved reactive oxidant species increasing from the metabolism. The liver injure caused by CCl₄ is due to the formation of a reactive toxic metabolite by the hepatic cytochrome P-450 system. As data of Table 3 indicated that lipoperoxides are obviously increased in 1, 3, 6, 12 and 24 hours and DFAD decreases lipoperoxides significantly.

### Example 3

### Effects of DFAD on Hepatic Microsomal Monooxygenases

As mentioned above section, the DFAD markedly decreased lipoperoxides. It means that DFAD could obviously protected injury, which caused by CCl₄.

In the present study, the effect of DFAD on the activities of hepatic microsomal monooxygenases was examined.

Microsomal preparations—the microsomes used were prepared from rats' liver. The liver was thoroughly perfused in situ with more than 200 ml of 0.9% NaCl solution. The liver was excised, and homogenized with 4 volumes of isotonic (1.15%) KCl solution in a Potter glass homogenizer. The homogenate was centrifuged at 12,000×g for 25 minutes in a refrigerated centrifuge, and the precipitate was discarded. The microsomes were sedimented by centrifugation at 78,000×g for 90 minutes in a Hitachi model 40P preparative ultracentrifuge. The firmly packed pellet of microsomes was resuspended in isotonic KCl solution with the Potter homogenizer and again centrifuged as above. The washed microsomes were finally suspended in isotonic KCl, usually at a concentration of 10 mg of protein per ml. The resultant microsomal suspensions were stored at 4° C. and used within 2 to 3 days. In these preparations isotonic KCl was employed, instead of the more usual 0.25 M sucrose, so as to minimize the adsorption of hemoglobin, when examined by zone elecrophoresis.

Cytochrome P-450, NADPH-cytochrome creductase, aminoyrine demethylase and benzpyrene hydroxylase determined as previously described. Other methods are as same as above section.

The experimental data are shown as the following table.

TABLE 4
Effect of DFAD on monooxygenases

| | Cytochrome | NADPH- | Aminopyrine | Benzpyrene |
|---|---|---|---|---|
| | P-450 | cytochrome | demethylase | hydroxylase |
| | (nmol/mg | Creductase | (nmol/mg | (nmol/mg |
| Group | protein) | (nmol/mg protein) | protein) | protein) |
| Control | 1.20 ± 0.13 | 120.8 ± 13 | 80.5 ± 9.0 | 20.8 ± 22 |
| DFAD | 1.50 ± 0.14* | 150 ± 16 | 95.0 ± 8.9 | 25.6 ± 3.8 |

*P < 0.05 as compared with control group.

The data of Table 4 indicated that DFAD increased the hepatic microsomal P-450, NADPH-cytochrome C, reductase aminopyrine demethylase, and benzopyrene hydroxylase activities. Above Results suggested that DFAD induced monooxygenases. It means that DFAD has a protective effect of acute hepatic injury.

### Example 4

### The Effect of DFAD on Control of Oncogenes

Human myeloblastic leukemic cell (ML-1) had been described previously. Cells were maintained in suspension culture in RPMI 1640 medium supplemented with 7.5% heat-inactivated FBS. Cells growth and viability were assayed by hemocytometer using trypan blue dye exclusion.

RNA was isolated by the CsCl gradient modification. RNA pellets were washed twice by reprecipitation in ethanol and quantitated by absorbency at 260 nM. RNA analyzed by electrophoresis of 15 μg of RNA through 1.2% agarose formaldehyde gels followed by northern blot transfer to nitrocellulose.

Single-standard uniformly labeled DNA probes were prepared. Probe of c-myc was a 1.7 Kb cla-Eco RI restriction fragment containing the 3′ exon region of human c-myc and probe of c-myc was 1.0 Kb myb-specific Bam HI fragment. Probes for n-ras contained DNA fragments using a modification of the PCR technique. Probes for myb, myc and n-ras were isolated by electrolution. The isolated fragments were labeled to high specific activity with [α³²P]-Dctp (3000 ci/mmol). Prehybridization of the filter was performed. The hybridization mixer contained 50,000 cpm of probe. The probes were hybridized at 58° C. and in 15 mM NaCl, 1.5 nM sodium citrate for 3 hours. After hybridization, they were exposed to XAR-5 film. Oncogene expression was quantitated by scanning of the autoradiography.

The Results are summarized in the tables as below.

TABLE 5
The effect of DFAD on inhibition of oncogenes

| Compound | Inhibition (%) | | |
|---|---|---|---|
| (ng/ml) | c-myb RNA | c-myc RNA | n-ras RNA |
| Cultured medium | 0 | 0 | 0 |
| DFAD | 25.6 ± 3.5 | 30.1 ± 4.2 | 35.7 ± 3.8 |

The Results presented above clearly suggested that DFAD has a significant effect of inhibiting oncogenes.

### Example 5

### Inhibition of Tyrodine Kinase Activity by DFAD

The development of cancer cells can be viewed as a defect in the normal process of differentiation and disorder balance between proliferation and maturation that occurs in normal cells. The expression of oncogenes plans a very important role in regulate cellular proliferation. The tyrosine kinase (TK) is a protein product of expression of oncogenes. The TK catalyze the transfer of phosphate from ATP to the hydroxyl residues on protein substrates. Activity of the TK is essential for the malignant transformation of cells.

Hunter and Sefton demonstrated that the protein product of the src oncogene was a TK. In subsequent years, a number of oncogenes have been found to code for TK. Such as src, yes, fgr, abl, erbB, mos, neu, fins, fps, ros and sis are considered to act through tyrosine kinase activity. TK activity is strongly correlated with the ability of retroviruses to transform cells. Also, maturation with reduced TK activity has lower transforming efficiency. Transformation of the HL-60 leukemia cells causes the high TK activity. In fact, TK activity is enhanced in many human cancers, such as breast carcinomas, prostate cancer cells, colon cancers, and skin tumor. The Results of a lot of experiments indicated that tyrosine phosphorylation is an important intracellular mediator of proliferation and differentiation. Mature of cells possess relatively low levels of TK activity. Similar TK activity is also related with the cellular receptors for several growth factors such as EGF, platelet-derived growth factor, insulin, and growth factor I.

In general, very low levels of TK are expressed in normal cells and high levels of TK are expressed in cancer cells. Many evidences have been accumulated that the dysfunction of cellular oncogenes is a cause of human cancers. Therefore, a drug, which inhibits the activity of TK, can provide a new way to overcome cancer. In other words, the development of effective inhibitors of TK can be used for the treatment of cancer.

### Materials and Methods

[³²P]ATP and other isotopes were purchased form Amersham Corp. All other chemicals were reagent grade obtained from commercial suppliers.

Cells: L1210 and P388 cells were grown at 37° C. on medium RPM-1640 without antibiotics and supplemented with 10% horse serum. Cultures were diluted daily to 1×10⁵ cells/ml with fresh growth medium. From a culture initiated with cells from ascitic fluid obtained from a mouse 5 days after implantation with in vivo-passage leukemia, a stock of ampoule containing 10⁷ cells/ml in growth medium plus 10% dimethyl sulfoxide was frozen and stored in liquid nitrogen. Cultures were started from the frozen stock and were passage for no more than 1 month.

L1210 and P388 cells were grown at 37° C. on medium RPMI-1640 supplemented with 10% calf serum, 10,000 unit/ml of Penicillin and 10,000 unit/ml of Streptomycin. 1×10⁶/ml cells were placed in culture with different concentrations of DFAD. Then the cell suspension was incubated at 37° C. in a humidified atmosphere of 5% CO₂-95% air for the indicated time. Reactions were terminated by addition of 3 ml of cold Earle's buffer. Cells were lysed, precipitated with 10% trichloroacetic acid (TCA) and filtered onto glass fiber filters. The filters were washed with phosphate-buffered saline and placed in scintillation vials, and radioactive emissions were counted.

Tyrosine kinase (TK) Assay: TK was measured by a modification of the method of Braun et al. Briefly, H-60 leukemia cells were plated at a density of 5×10⁵ cells in 60-nm dished, and divided control and treatments groups for incubation 24 hours at 37° C. with 5% CO₂. The cells were collected by scraping, washed twice with phosphate-buffered saline, and resuspended at density of 10⁶ cells/ml in 5 mM HEPEs buffer (pH 7.4). The cells were then resuspended in 1 ml of buffer containing 5 mM HEPES (pH 7.6), 1 mM MgCl₂ and 1 mM EDTA, then placed on ice bath. The cell membrane was disrupted by ultra sound and centrifuged at 1000×g for 10 minutes. The supernatant was ultra centrifuged at 30,000×g for 30 minutes at 4° C. The pellet was resuspended in 0.3 ml of buffer containing 25 mM HEOES, centrifuged at 12,000×g for 5 minutes. The resulting supernatant was used for TK assay. Content of protein was determined. 10 μg of protein placed in 20 mM HEPES (pH 7.6), 15 mM MgCl₂, 10 mM ZnCl₂ and 5% (v/v) nonidet P-40, with or without substrate [glutamic acid (GT), mg/ml]. After 5 minutes incubation at 25° C., the reaction was initiated by the addition of 25 μM [γ³²P] ATP (3 ci/mmol). After 10 minutes, the reaction was stopped by the addition of 20 mM cold ATP. 50 μl of the mixtures were spotted on glass microfiber filter discs and washed three times with cold trichloroacetic acid (TCA), contained 10 mM sodium pyrophosphate. Air dried. Radioactivity was determined by liquid scintillation spectrometry. The net TK activity was determined after correcting for endogenous TK activity.

### Results and Discussion

The present study clearly demonstrated that DFAD reduction in TK activity.

TABLE 6
Effect of DFAD on TK activity of HL-60 leukemia cells

| Drugs | % of control activity |
|---|---|
| Control | 100 |
| DFAD | 85.6 ± 9.0* |

*P < 0.05 as compared with control group.

Value represents the mean of three experiments each done in duplicate; the range was less than 5% of the mean.

Value represents the mean of three experiments each done in duplicate; the range was less than 5% of the mean.

### Example 6

### Mutagenic Effect of DFAD

Determination of the mutagenic and carcinogenic activity is important for estimating side effects of drug. The mutagenic activity of many drugs can only be detected with growing cells. In present study, mutagenic and carcinogenic activity of DFAD is determined by Bacteria system.

The method for detecting mutagenicity of DFAD, with the Salmonella system that detects the reversion of the bacteria from His⁻ to His⁺, is widely used.

Methods for detecting carcinogens and mutagens with the salmonellia mutagenicity test are highly efficient in detecting carcinogens and mutagens. Major carcinogens tested have been detected as mutagens. Salmonella mutagenicity assay is very sensitive and simply test for detecting mutagens and carcinogens. Therefore, it has been useful in a detailed study that has been made of mutagenic activity of DFAD.

TAa7, TAa8, TA100 and TA102, which developed by Ames et al, are extremely effective in detecting classes of carcinogens and mutagenesis.

### Methods

The bacterial tester strains used for mutagenesis testing are TA97, TA98, TA100 and TA102. Mutagenesis testing method was done as described previously. In brief, TA97, TA98, TA100 and TA102 were grown in agar gel culture. The petri plats (100×15 mm style) contain 30 ml of vogelbonner medium with 2% glucose. The agar mixture was agitated vigorously and immediately poured into plates of minimal agar. The cultures were incubated at 37 in a dark and 5% CO₂ in air for 48 hours. After 48 hours the colonies (revertants to histidine prototrophy) in both test and controls are counted. The presence of a background lawn of bacteria on the histidine-poor soft agar plate was used as an indication that gross toxic effects were absent. Mutagenicity assays were carried out at least in triplicate.

### Results and Discussion

The data of experiment summarized as the following table.

TABLE 7
Mutagenesis Assay on plates

| | Dose/ | Number of His⁺ revertants/plate | | |
|---|---|---|---|---|
| | plate | TA97 | TA98 | TA100 |
| Treatment | (μg) | —S | —S | —S |
| Spontaneous | — | 149 ± 15 | 35 ± 4 | 120 ± 17 |
| 4NQO | 0.5 | 861 ± 79 | 338 ± 35 | 2301 ± 190 |
| DFAD | 100 | 260 ± 21* | 120 ± 19* | 250 ± 26* |

4QO: 4-nitroquinoline-1-Oxide
*P < 0.05 as compared with control group.

The salmonella typhimurium strains TA97, TA98 and TA100 were checked using 4-nitroquinoline-1-oxide. The range of spontaneous mutation rates for the individual strains, which were considered to be acceptable, was TA97 (100-170), TA98 (20-40) and TA100 (80-150).

The data of Table 7 indicated that DFAD is not a carcinogenic and mutagenic agent.

### Example 7

The effect of DFAD on lymphoblastoid transformation by means of ³H-TdR liquid scintillation assay technique was investigated.

(1) Male mice weight 18-20 were used in the experiments. They were divided into three groups: normal, immunosuppressed and immunosuppressed + DFAD. The DFAD dosage of is 5.5 mg/kg was injected intraperitoneally to each of the mice in the immunosuppressed + DFAD group. The normal mice were injected with same volume of normal saline. These injections were repeated daily for 3-5 days. On the last day, both immunosuppressed and immunosuppressed + DFAD groups were injected interapertioneally with one of the immunosuppressive agents including cortisone, cyclosporin A, prednisone, azathioprine, mercaptopurine, vincristine or chlorambucil. The experimental procedure for all the examples with mice is similar to the above procedures.

### (2) Lymphoblastoid Transformation Test:

### I. Reagents and Conditions for Cell Culture

a. Culture media—RPMI 1640, madium 199 minimal essential medium (Eagle).
b. 37° C. to maintain the pH of the medium at 7.31.
c. Serum—generally 15-20% fetal bovine serum was incorporated, for lymphocytes from mice, 5% was used.
d. Gaseous phase 5% CO₂ in air.
e. Cell concentration—generally 1-2×10/ml
f. Stimulants—20 μl/ml for phytoagglutinin containing polysaccharide (PHA-M) or 10 μ/ml for polysaccharide-free purified phytoagglutinin (PHA-P).

### II. Measured by Liquid Scintillation

a. The conditions of cell culture are same as above. ³H-TdR is added after 48 hours of incubation at a final concentration of 2 μCi/ml and continue the incubation for 24 hours.
b. Wash the cells twice with cold normal saline and lyse the erythrocytes by addition of distilled-water and equal volume of 3.6% NaCl was the added. Wash again the intact lymphocytes once with cold saline. Spin down the lymphocytes and add 2 ml of 10% trichloroacetic acid to precipitate the protein. Wash twice the normal saline. Add 2 ml of ethanol:ether (1:1) to wash once. 0.2 ml of formic acid is then added for digestion till the precipitate is dissolved.
c. Add 4 ml of scintillation fluid to 0.1 ml of the final sample and count in a liquid scintillation counter.
(3) Results are listed in the following tables:

TABLE 8

| | | | Immunosuppressed + |
|---|---|---|---|
| | Normal | Immunosuppressed | DFAD |
| CPM | 1340 ± 51 | 620 ± 58 | 1286 ± 54 |
| Number | 10 | 10 | 10 |
| of sample | | | |
| P | — | | <0.01 |

*CPM: count per minute

### Example 8

### The Influence of DFAD on Formation of Rosette in Guinea Pigs

### I. Method

1. Obtain venous blood in heparin (10 IU ml⁻¹) and perform a total and differential leucocyte count.
2. Isolate lymphocyte fraction. Count viable lymphocytes calculate and record total yield. Adjust to 5×10⁶ lymphocytes ml⁻¹.
3. Wash sheep erythrocytes by centrifugation (400 g for 10 min. At room temperature) and adjust to a 2.5% v/v suspension in PBS.
4. Mix 0.1 ml of the lymphocyte suspension with 0.1 ml of sheep erythrocytes and centrifuge at 225 g for 5 min. at room temperature.
5. Incubate for 2 hours at 4° C.
6. Add 50 μl of fetal bovine serum (FBS) and 50 μl of nigrosin solution.
7. resuspend cell mixture by gently tapping the tube and pipette a sample into a haemocytomer.
8. Count 200 lymphocytes and determine the percentage of cells with 3 or more erythrocytes attached. (These are T lymphocytes)
9. Calculate the absolute number of T lymphocytes ml⁻¹ of original blood.
II. Results are listed in the following table:

TABLE 9

| | | | Immunosuppressed + |
|---|---|---|---|
| | Normal (%) | Immunosuppressed | DFAD |
| Rate of | 43.8 ± 2.0 | 20.7 ± 1.8 | 41.4 ± 3.6 |
| formed | | | |
| rosette | | | |
| Number | 12 | 12 | 12 |
| of sample | | | |
| P | — | | <0.01 |

### Example 9

### The Effect of DFAD on Phagocytosis of Peritoneal Macrophage of Mice

### ⁵³Cr Labeling Method:

Method—Count the number of macrophages in the peritoneal exudate of mice and adjust to 1×cell⁷/ml with normal saline. Add 0.1 ml of the macrophage suspension i.e. 1×10⁶ cells to each well on the plastic plate for test. Label the chick red blood cell with ⁵³Cr. Suspend the labelled chick red blood cell and adjust to 1.5×10⁸/ml, add 0.1 ml, thereof i.e. 1.5×10⁷, to each well. Incubate at 37° C. for 30 minutes. Wash thoroughly to remove the free chick red blood cells. Count each well in a Y-counter.

Results are listed bellow.

TABLE 10

| | | | Immuno-suppress + |
|---|---|---|---|
| | Normal | Immuno-suppressed | DFAD |
| CPM | 1089 ± 341 | 481 ± 44 | 908 ± 72 |
| Number of | 12 | 12 | 12 |
| samples | | | |
| P | — | | <0.1 |

### Example 10

### Influence of DFAD on Complement

Complement is a group of normal serum proteins. When the body is invaded by pathogenic microorganisms, complement acting together with specific antibodies exhibits its defensive function. It plays an important role in the anti-infectious immunity of the body. In addition, the complement system can also be activated by bacterial before the production of antibody by the body and achieves its bacteriocidal effect and inactivates the virus through the by-path.

### 1. Materials:

a. Buffer Stock:
NaCl 85.00 g. Barbituric acid 5.75 g. Sodium barbital 3.75 g. Add 1500 ml of distilled water and heat to dissolve, add distilled water to 2000 ml.
b. 0.1M EDAT—Na stock:

### EDTA—Na.sub.3 37.23 g, NaOH 4.00 g

Add the EDTA—Na.sub.3 to 500 ml of distilled water and the NaOH to 100 ml of distilled water. Add the later to the former and EDTA—Na.sub.3 will dissolve instantly.
Adjust PH to 7.5 with in NaOH and add distilled water to 1000 ml.
c. 2% gelatin:
Gelatin 2.0 g, distilled water 100 ml, heat to dissolve and store at 4° C.
d. Gelatin Veronal Buffer (GVB)
Buffer stock: 100 ml

### 0.03M CaCl: 10 ml

### 0.01M MgCl: 10 ml

### 2% Gelatin: 100 ml

Add distilled water to: 1000 ml
e. Alsever solution:
Glucose 20.5 g, NaCl 4.2 g, Sodium citrate 8.0 g dissolve in approximately 800 ml of distilled water and adjust PH to 6.1 with citric acid. Add distilled water to 1000 ml.
Sterilize by autoclaving.

### f. 0.01M EDTA—GVB:

Buffer stock 360 ml, 0.1M EDTA—Na.sub.3 stock 200 ml, 2% Gelatin 100 ml, add distilled water to 2000 ml.
g. SRBC:
Mix fresh sterile sheep blood with equal volume of Alsever solution and store at 4° C. It can be used for several weeks.
h. Hemolysin:

### (1) Preparation of SRBC Stroma:

Spin down the SRBC in liter of sheep blood—alsever solution and wash several times with normal saline. Add 10 liters of distilled water which contains 4 ml of glacial acetic acid. Suspend the RBC and let it sit in a 4° C. refrigerator overnight. Discard the supernatant and pack the settled stroma at 2,000 rpm. Suspend the stroma in 0.01M acetic acid solution. The acetic acid was then removed and the PH brought to neutral or slightly alkaline by wash the stroma 3 times each with 0.1 M Na₂ HPO₄ and normal saline. Pack the stroma by spinning at 7,500 rpm. The packed SRBC stroma was then suspended in 300-400 ml of normal saline. Heat to 100° C. for 1 hour. Determine the nitrogen content and adjust with sterile normal saline to 1 mg/ml. Add 0.01% merthiolate and store at 4° C.

### (2) Immunization of Rabbits:

Immunize the rabbits by 11 intravenous injections of the SRBC stroma in 2 weeks. Bleed the animals 4 days after the last injection. Separate the serum. Inactivate at 56° C. for 30 minutes and store at −20° C.

### (3) Titration for Optimal Concentration of Hemolysin:

By using 50% hemolysin (C′H₅₀) as end-point, SRBC sensitized by various concentrations of hemolysin were titrated against various amounts of guinea pig complement. Optimal concentration of hemolysin was determined by OD₅₄₁ reading which gave C′H₅₀ and standard curve plotted.

i. Serum Samples for Determination of Complement Content.

### 2. Methods:

a. Preparation of SRBC suspension—wash SRBC for 5 times with GVB to free from platelets. Filter through gauze to remove cell aggregates.
b. Preparation of sensitized SRBC—warm up 1 volume of hemolysin at the optimal concentration in a 37° C. water bath for 10 minutes and add equal volume of SRBC suspension at 1×10⁹ cells/ml with stirring. Let it sit in a water bath at 37° C. with shaking for 30 minutes. Then bring the temperature down in a ice-cool water bath shaking. Wash the cold SRBC once with 0.01M EDTA—GVB, twice with GVB and prepare sensitized SRBC suspension at 5×10⁸ cells/ml with GVB.
c. Determination of C′H₅₀ unit and plotting of standard curves for the serum samples.

TABLE 11

| | Number of sample | Units (C′H₅₀)/ml |
|---|---|---|
| Normal | 18 | 509 ± 10 |
| Immunosuppressed | 18 | 219 ± 9 |
| Immunosuppressed + DFAD | 18 | 289 ± 18 |
| P | | <0.05 |

The data of table 11 indicated that DFAD increases complement of serum.

1-41. (canceled)

42. A botanical drug which contains Dichroa febrifugine (DF) has new pharmaceutical function including antioxidation, control oncogen and increase immune function.

43. A botanical drug of claim 42 wherein DF has a function of inhibiting super-oxidation.

44. A botanical drug of claim 42 wherein DF has a function of inhibiting peroxidation.

45. A botanical drug of claim 42 wherein DF has a function of inhibiting microsomal monooxygenases.

46. A botanical drug of claim 42 wherein DF has a function of controlling oncogen.

47. A botanical drug of claim 42 wherein DF has a function of inhibiting tyrosine kinase (TK) activity.

48. A botanical drug of claim 42 wherein DF has a function of increasing lymphoblastoid transformation.

49. A botanical drug of claim 42 wherein DF has a function of increasing formation of rosette of lymphocytes.

50. A botanical drug of claim 42 wherein DF has a function of increasing phagocytosis of macrophage.

51. A botanical drug of claim 42 wherein DF has a function of increasing complement of serum.

52. A botanical drug, which contains Dichroa febrifugine (DF), is a safe composition.

53. Dichroa febrifugine (DF) is extracted from Dichroa febrifuga lour.
